(19) United States
(12) Patent Application Publication
(10) Pub. No.: US 2026/0235566 A1
(43) Pub. Date: Aug. 13, 2026

(54) GAS SENSOR

(71) Applicant: Asahi Kasei Microdevices Corporation, Tokyo (JP)

(72) Inventors: Keiichiro KUWATA, Tokyo (JP); Takaaki FURUYA, Tokyo (JP)

(73) Assignee: Asahi Kasei Microdevices Corporation, Tokyo (JP)

(21) Appl. No.: 19/451,135

(22) Filed: Jan. 16, 2026

(30) Foreign Application Priority Data
Feb. 7, 2025 (JP) 2025-019623
Oct. 27, 2025 (JP) 2025-181031

(51) Int. Cl. G01N 33/00; G01N 21/61

(52) U.S. Cl. CPC G01N 33/0027; G01N 21/61; G01N 2201/022; G01N 2201/062; G01N 2201/0636

(57) ABSTRACT

A gas sensor that includes a substrate and a light-guiding member 5. The light-guiding member 5 includes a first reflector 51 and a second reflector 52 that reflect at least a portion of light emitted by a light emitter, and is integrally formed to include the first reflector 51 and the second reflector 52. The first reflector 51 and the second reflector 52 are arranged to face each other in a plan view of the gas sensor. The light-guiding member 5 includes a strain absorber 5a between the first reflector 51 and the second reflector 52 in the plan view, which is capable of absorbing strain generated in the substrate and/or the light-guiding member 5.

### CROSS-REFERENCE TO RELATED APPLICATION

The present application claims priority to and the benefit of Japanese Patent Application No. 2025-019623 filed February 7, 2025, and Japanese Patent Application No. 2025-181031 filed October 27, 2025, the entire contents of which are incorporated herein by reference.

### TECHNICAL FIELD

The present disclosure relates to a gas sensor.

### BACKGROUND

Conventionally, there are known gas detection devices (gas sensors) that include a substrate provided with a light-emitting element (light emitter) that emits light and a light-receiving element (light receiver) that receives the light on a main surface thereof, and a light-guiding member that is joined to the substrate on the main surface side of the substrate and guides the light emitted by the light-emitting element (light emitter) to the light-receiving element (light receiver) (for example, see Patent Literature (PTL) 1). In the gas sensor described in PTL 1, the substrate and the light-guiding member are joined, for example, via only a joining member that serves as an axis of rotation when the light-guiding member moves relative to the substrate (that is, connection is at only one point). Therefore, even when strain occurs in the substrate due to thermal expansion, the light-guiding member is not affected by that deformation, and as a result, distortion of an optical path of the gas sensor is suppressed, and changes in gas detection sensitivity are reduced.

### CITATION LIST

### Patent Literature

PTL 1: JP 2021-144027 A

### SUMMARY

### Technical Problem

In the gas sensor described in PTL 1, fixing strength between the substrate and the light-guiding member is weak, and therefore, when vibrations or the like are applied, a precisely adjusted optical system may collapse, which may ultimately result in a degradation of gas detection performance. It has been found that there is room for further improvement in terms of suppressing strain in the gas sensor.

It would be helpful to provide a gas sensor that can suppress degradation of gas detection performance due to strain.

### Solution to Problem

Means for achieving the above are as follows:

(1) A gas sensor of the present disclosure comprises:

a substrate comprising, on a main surface thereof, a light emitter configured to emit light and a light receiver configured to receive at least a portion of the light emitted by the light emitter; and

a light-guiding member joined to the substrate on the side of the main surface of the substrate and configured to guide at least a portion of the light emitted by the light emitter to the light receiver, wherein

the light-guiding member comprises a first reflector and a second reflector that each reflect at least a portion of the light emitted by the light emitter, and is integrally formed including the first reflector and the second reflector,

the first reflector and the second reflector are disposed to face each other in a plan view of the gas sensor, and

the light-guiding member comprises a strain absorber between the first reflector and the second reflector in the plan view, the strain absorber being capable of absorbing strain generated in the substrate and/or the light-guiding member.

(2) In the gas sensor according to (1),

the first reflector and the second reflector are preferably configured to be separated from each other by the strain absorber.

(3) In the gas sensor according to (1) or (2),

the strain absorber is preferably configured as a thin portion that has a wall thickness thinner than a portion of the light-guiding member surrounding the strain absorber.

(4) In the gas sensor according to any one of (1) to (3),

a through hole is preferably provided in the strain absorber.

(5) In the gas sensor according to any one of (1) to (4),

a light-emitting element constituting the light emitter may be an LED.

(6) In the gas sensor according to any one of (1) to (5),

the light-guiding member and the substrate are preferably fixed by a plurality of fixing portions.

(7) In the gas sensor according to (6),

at least two of the plurality of fixing portions are preferably arranged to sandwich the strain absorber in a plan view of the gas sensor.

(8) In the gas sensor according to any one of (1) to (7),

the light-guiding member may be an imaging optical system configured to form an image at the light receiver with at least a portion of the light emitted by the light emitter.

(9) In the gas sensor according to (8),

in the imaging optical system of the light-guiding member, a size of the image formed may be smaller than an area of a light-receiving surface of the light receiver.

(10) In the gas sensor according to any one of (1) to (9),

the gas sensor may be a surface mounted type.

### Advantageous Effect

According to the present disclosure, it is possible to provide a gas sensor that can suppress degradation of gas detection performance due to strain.

### BRIEF DESCRIPTION OF THE DRAWINGS

In the accompanying drawings:

FIG. 1 is a perspective view diagram of a gas sensor according to a first embodiment of the present disclosure, as viewed obliquely from above;

FIG. 2 is a perspective view diagram of a light-guiding member included in the gas sensor of FIG. 1, as viewed obliquely from above;

FIG. 3 is a perspective view diagram of the light-guiding member of FIG. 2, as viewed obliquely from below;

FIG. 4 is a schematic perspective view diagram of the gas sensor of FIG. 1, with a portion of the gas sensor being transparent, for explaining an optical path in the gas sensor;

FIG. 5 is a schematic explanatory diagram of a cross-section taken along line A-A in FIG. 1, for explaining a state of a strain absorber in the gas sensor of FIG. 1;

FIG. 6 is a perspective view diagram of a light-guiding member included in a gas sensor according to a second embodiment of the present disclosure, viewed as in FIG. 3;

FIG. 7A, FIG. 7B, and FIG. 7C are diagrams illustrating a test article of a comparative example used in simulation to confirm the disclosed effects, where FIG. 7A is a perspective view diagram of the test article seen obliquely from above, FIG. 7B is a perspective view diagram of the test article seen obliquely from below, and FIG. 7C is a cross-section diagram taken along the line B-B of FIG. 7A;

FIG. 8A, FIG. 8B, and FIG. 8C are diagrams illustrating a test article of an example used in simulation to confirm the disclosed effects, where FIG. 8A is a perspective view diagram of the test article seen obliquely from above, FIG. 8B is a perspective view diagram of the test article seen obliquely from below, and FIG. 8C is a cross-section diagram taken along the line C-C of FIG. 8A; and

FIG. 9 is a graph illustrating results of simulations for confirming the disclosed effects.

### DETAILED DESCRIPTION

A gas sensor according to an embodiment of the present disclosure will now be described with reference to the accompanying drawings.

Members and sites common to each drawing are marked with the same reference signs. The drawings are schematic, and dimensions and proportions of each member and portion, and dimensional relationships or proportions between the drawings, may not be to scale.

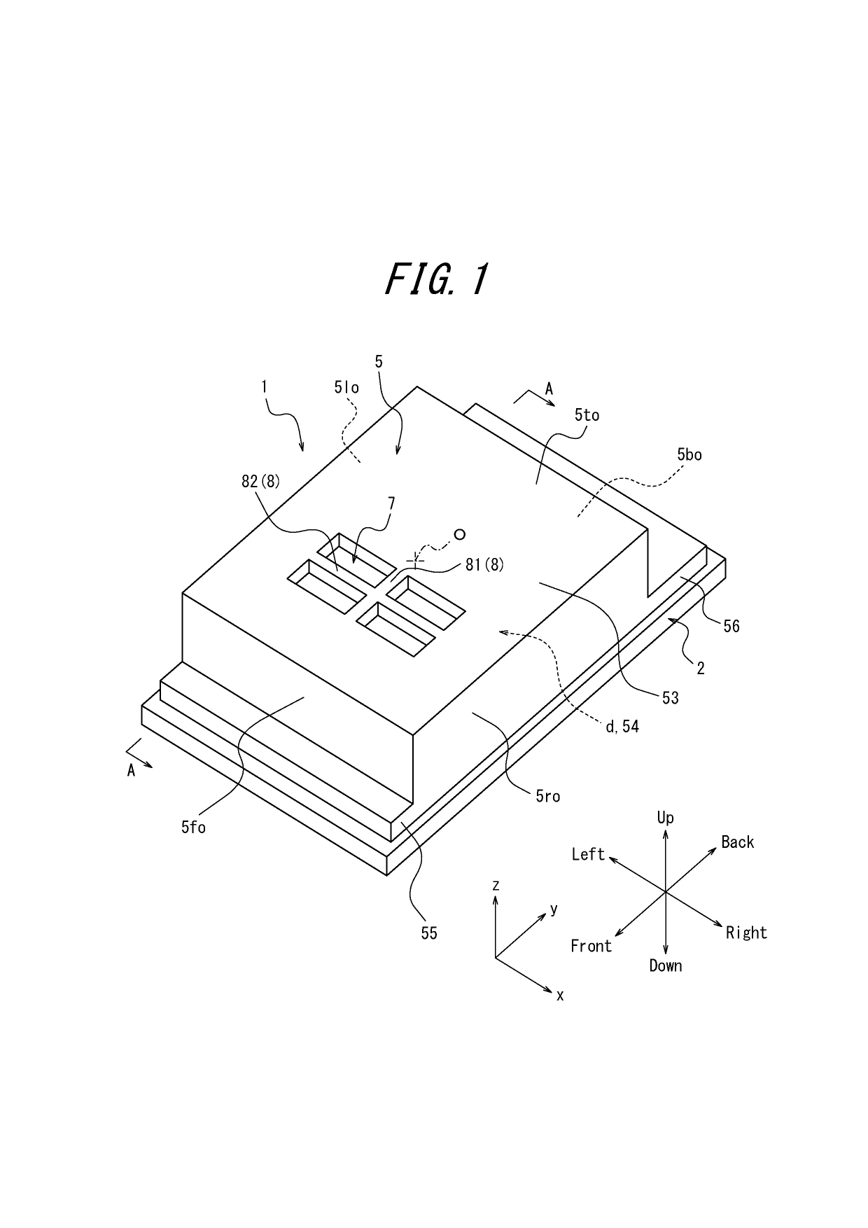

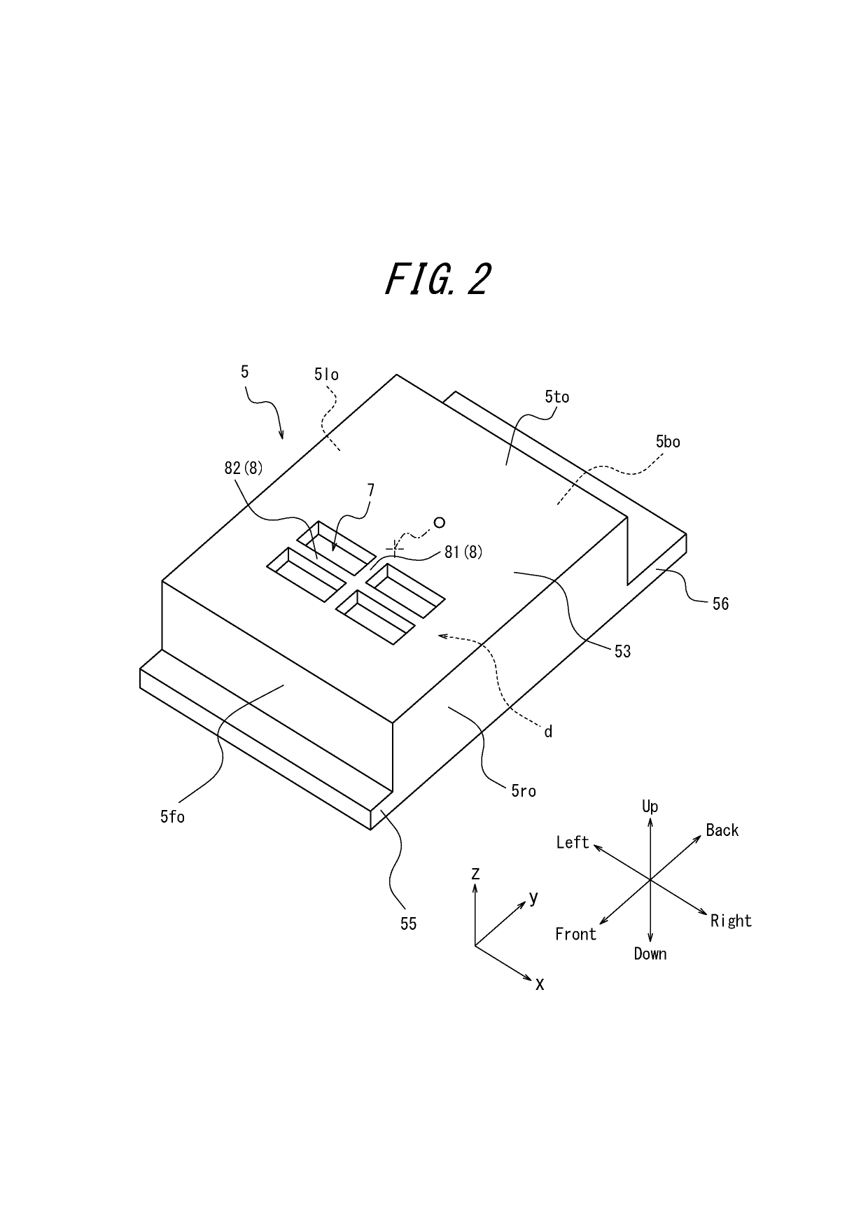

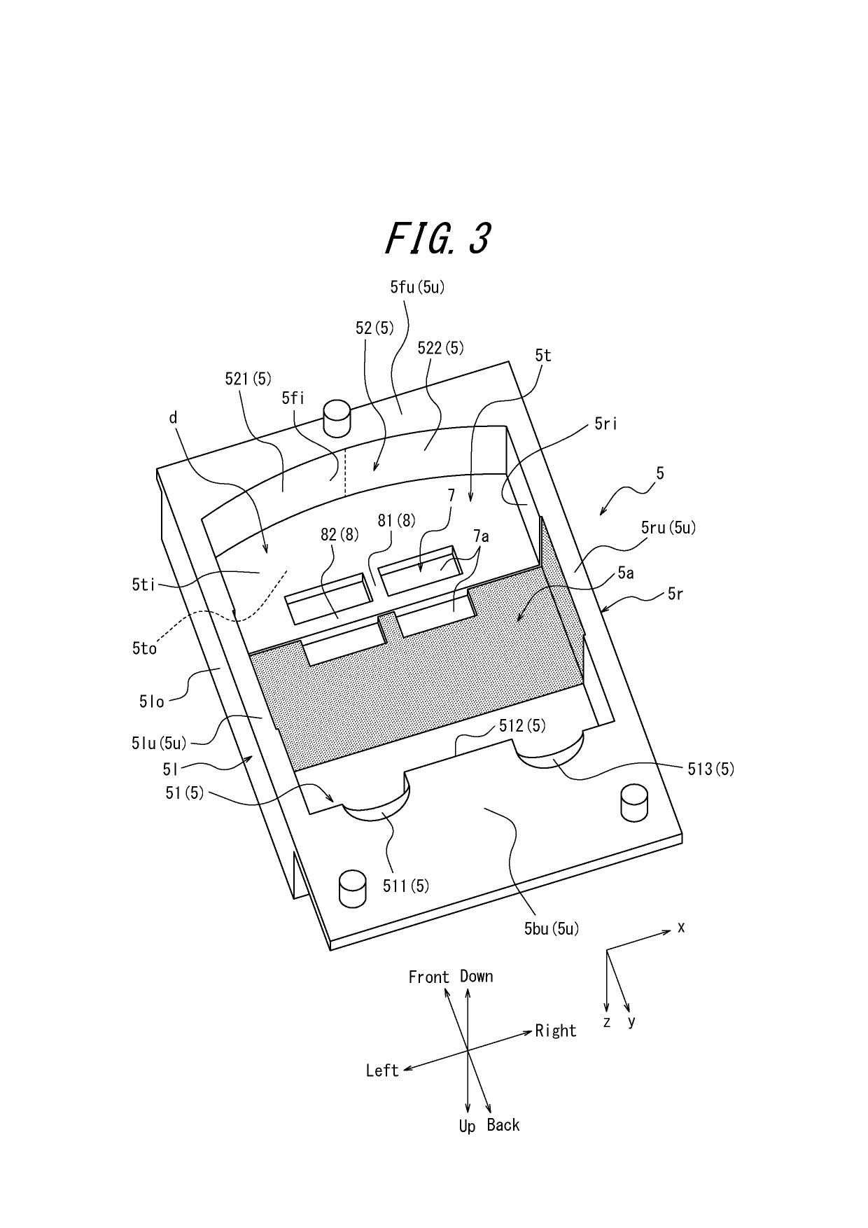

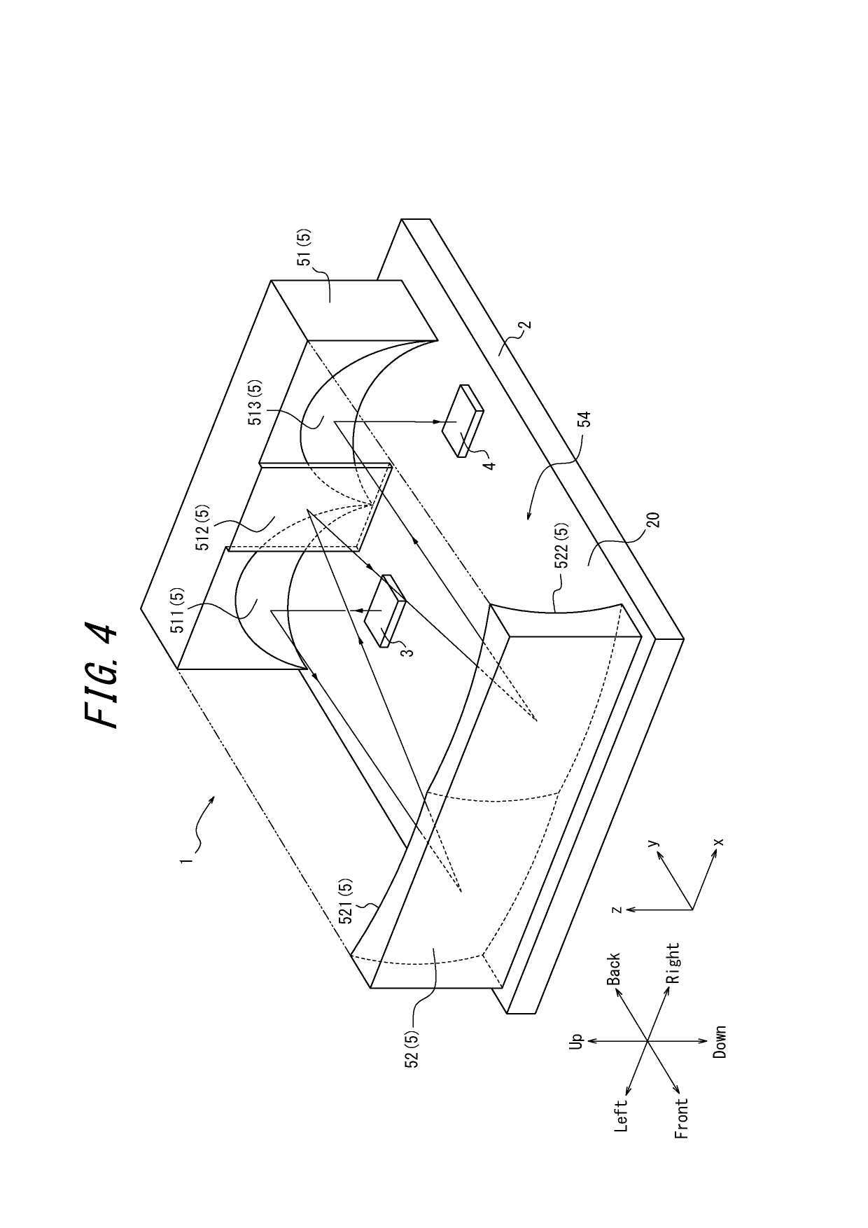

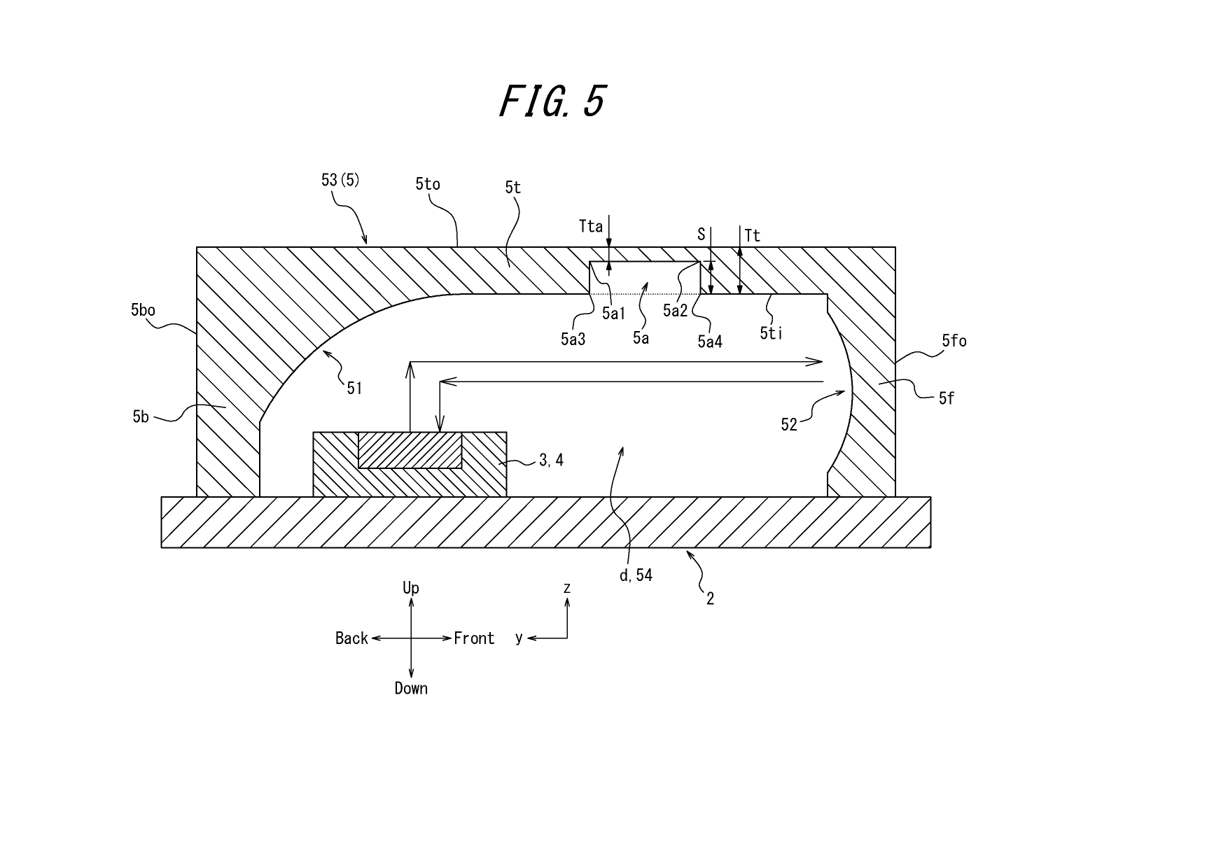

FIG. 1 is a perspective view diagram of a gas sensor according to a first embodiment of the present disclosure, as viewed obliquely from above. FIG. 2 is a perspective view diagram of a light-guiding member included in the gas sensor of FIG. 1, as viewed obliquely from above. FIG. 3 is a perspective view diagram of the light-guiding member of FIG. 2, as viewed obliquely from below. FIG. 4 is a schematic perspective view diagram of the gas sensor of FIG. 1, with a portion of the gas sensor being transparent, for explaining an optical path in the gas sensor. FIG. 5 is a schematic explanatory diagram of a cross-section taken along line A-A in FIG. 1, for explaining a state of a strain absorber in the gas sensor of FIG. 1. However, for the sake of simplicity, the gas sensor 1 is illustrated in FIG. 1 without illustrating the inside of the light-guiding member 5, such as the light emitter 3 and the light receiver 4 illustrated in FIG. 4, which could be seen through a ventilation opening 7. Similarly, in FIG. 2, the inside of the light-guiding member 5 visible through the ventilation opening 7 is not depicted. Further, in FIG. 4 and FIG. 5, a front foot portion 55 and a back foot portion 56 (see FIG. 1 and FIG. 2) of the light-guiding member 5, which are described later, are omitted from the illustration. FIG. 5 is merely a schematic explanatory diagram and does not accurately represent a cross-section taken along line A-A in FIG. 1.

According to the present embodiment, the gas sensor 1 is a small device having dimensions of, for example, 30 mm ´ 20 mm ´ 10 mm. According to the present embodiment, the gas sensor 1 is a non-dispersive infrared (NDIR) gas detection device that measures concentration of a gas to be detected based on infrared rays transmitted through an introduced gas body. As another example, the gas sensor 1 may be a photoacoustic gas detection device. The gas to be detected may be, for example, carbon dioxide, water vapor, methane, ethane, propane, butane, formaldehyde, carbon monoxide, nitrogen monoxide, ammonia, sulfur dioxide, alcohol (methanol, ethanol, or the like), a chlorofluorocarbon, a hydrochlorofluorocarbon, a hydrofluorocarbon, a refrigerant gas (R32, R290, or the like), or a mixture of these.

The configuration of the gas sensor 1 according to the present embodiment allows use as a light emitting and receiving device for applications other than gas detection. That is, disclosure obtained by replacing the term "gas sensor" described in the specification and claims of this application with "optical concentration measuring device", "optical physical quantity measuring device", "light receiving and emitting device", "optical device", or the like, is included in the scope of the present disclosure. For example, the state of an optical path space can be detected (examples other than gas include the presence or absence or concentration of a specific component of a fluid). For example, the disclosure content can be used for a component detection device or a component concentration measuring device for a substance (for example, water or a body fluid) present in an optical path space between the light emitter and the light receiver. For example, when the substance present in the optical path space is blood, the component detection device or the component concentration measuring device can be used to measure glucose concentration in blood.

The component detection device or the component concentration measuring device can measure glucose concentration in blood by measuring absorption of light having a wavelength of 1 mm to 10 mm. In the measurement of glucose concentration in blood, measuring absorption of light at 1.6 mm, 2.0 mm to 2.3 mm, and 9.6 mm is preferred. A compact, high precision, and highly reliable non-invasive glucose concentration meter can be realized. Such a glucose concentration meter allows, for example, a diabetic patient to self-check blood sugar levels with good precision and without causing damage to the skin as would occur with an invasive method. Further, more accurate administration of medication (for example, insulin) can be achieved, based on the blood sugar levels checked.

As illustrated in FIG. 1 and FIG. 4, the gas sensor 1 includes a substrate 2, a light emitter 3, a light receiver 4, and a light-guiding member 5. FIG. 4 illustrates an example configuration of the gas sensor 1 with a portion of the light-guiding member 5 being transparent, where the light emitter 3 and the light receiver 4 on a main surface 20 of the substrate 2 are visible. According to the present embodiment, the main surface 20 is a surface among those having the largest area of the substrate 2 on which the light-guiding member 5 is disposed. The light-guiding member 5 in the present embodiment is the light-guiding member 5 in some embodiments described in detail later.

Hereinafter, as illustrated in each of FIG. 1 to FIG. 6, for convenience of explanation, Cartesian coordinates are set so that the xy plane is parallel to the main surface 20 of the substrate 2. The z-axis direction is perpendicular to the main surface 20 of the substrate 2. The x-axis direction and the y-axis direction are parallel to sides of the main surface 20 of the substrate 2. Here, the y-axis direction corresponds to the direction in which a first reflector 51 and a second reflector 52 described later face each other, and the direction from the second reflector 52 to the first reflector 51 is defined as the positive y-axis direction.

Further, hereinafter, unless otherwise specified, in the Cartesian coordinates indicated in each drawing, the y-axis direction is sometimes referred to as the front-back direction, the negative y-axis direction (negative side) as the front (front direction, front side), and the positive y-axis direction (positive side) as the back (back direction, rear side, back side). Further, the x-axis direction is sometimes referred to as the left-right direction, the negative x-axis direction (negative side) as the left (left direction, left side), and the positive x-axis direction (positive side) as the right (right direction, right side). Further, the z-axis direction is sometimes referred to as the up-down direction, the positive z-axis direction (positive side) as the top (upward direction, upper side), and the negative z-axis direction (negative side) as the bottom (downward direction, lower side). Hereinafter, the term "plan view" refers to an overhead view (including a transparent view) of the light-guiding member 5 and thus the gas sensor 1 from outside along the z-axis direction, in either the positive z-axis direction (upward) or the negative z-axis direction (downward). Further, "side view" refers to viewing (including a transparent view) the light-guiding member 5 and thus the gas sensor 1 from outside along the x-axis direction in either the negative x-axis direction (left direction) or the positive x-axis direction (right direction).

The substrate 2 is a plate-like member on which components of the gas sensor 1 are mounted and on which mounted electronic components are electrically connected. The substrate 2 may be, for example, a printed circuit board in which conductive wiring is printed on a plate made of glass epoxy resin or the like. Further, the substrate 2 may be, for example, a ceramic substrate or a flexible printed circuit board. The substrate 2 has the light emitter 3 including a light-emitting element and the light receiver 4 including a light-receiving element provided on the main surface 20 thereof. That is, the substrate 2 has, on the main surface 20, the light emitter 3 that emits light and the light receiver 4 that receives at least a portion of the light emitted by the light emitter 3. The substrate 2 may further have other electronic components mounted thereon. For example, the substrate 2 may be provided with a controller that controls at least one of the light emitter 3 or the light receiver 4 on the main surface 20 or on a bottom surface that is the opposite side to the main surface 20. The controller may include an analog front-end (AFE) or an analog-to-digital converter (ADC). Further, the substrate 2 may be provided with an arithmetic unit on the main surface 20 or on the bottom surface to execute arithmetic operations in gas concentration calculation. The arithmetic unit may include at least one general-purpose processor that executes functions according to a program to be read and may include at least one dedicated processor specialized for a particular process. The dedicated processor may include an application specific integrated circuit (ASIC). The processor may include a programmable logic device (PLD) or a microcontroller unit (MCU). The arithmetic unit may be integrated with the controller described above.

The light emitter 3 (more specifically, the light-emitting element that constitutes the light emitter 3) is a component that emits light used to detect gas to be detected. The light emitter 3 is not particularly limited as long as the light emitter 3 outputs light including a wavelength that is absorbed by the gas to be detected. According to the present embodiment, the light emitted by the light emitter 3 is infrared light, but is not limited to this example. According to the present embodiment, the light emitter 3 is a light-emitting diode (LED), but other examples may include a semiconductor laser, an organic light emitter, a micro-electromechanical systems (MEMS) heater, a light bulb, and the like. The light emitter 3 is provided in a defined region on the main surface 20 of the substrate 2. The defined region is determined to be a position facing a first mirror 511 (described later) in the z-axis direction. The light emitter 3 may include an optical filter having a wavelength selection function or a lens having a light condensing function.

The light receiver 4 (more specifically, the light-receiving element that constitutes the light receiver 4) is a component that receives light that has passed through a gas body introduced into a cell 54, which is described later. The light receiver 4 is not particularly limited as long as the light receiver 4 is sensitive to a band of light that includes a wavelength absorbed by the gas to be detected. According to the present embodiment, light received by the light receiver 4 is infrared light, but is not limited to this example. According to the present embodiment, the light receiver 4 is a photodiode, but other examples include a phototransistor, a thermopile, a pyroelectric sensor, a bolometer, and the like. The light receiver 4 converts received light into an electrical signal and outputs the converted electrical signal. The electrical signal is output to, for example, an arithmetic unit. Upon receiving the electrical signal, the arithmetic unit calculates the concentration of the gas to be detected, based on light transmittance and the like. The light receiver 4 is provided in a defined region on the main surface 20 of the substrate 2. The defined region is determined to be a position facing a fifth mirror 513 (described later) in the z-axis direction. The light receiver 4 may include an optical filter having a wavelength selection function or a lens having a light condensing function. Further, when the gas sensor 1 is a photoacoustic gas detection device, the gas sensor 1 may include a microphone instead of the light receiver 4.

The light-guiding member 5 is a member that guides at least a portion of the light emitted by the light emitter 3 to the light receiver 4. The light-guiding member 5 is an optical system of the gas sensor 1. The light-guiding member 5 includes optical members and configures the optical path from the light emitter 3 to the light receiver 4. In other words, the light-guiding member 5 optically connects the light emitter 3 and the light receiver 4. Here, the optical members include, for example, mirrors, lenses, and the like. The light-guiding member 5 is joined to the substrate 2 on the main surface 20 side of the substrate 2.

According to the present embodiment, the light-guiding member 5 includes the first reflector 51 and the second reflector 52. More specifically, the light-guiding member 5 includes the first reflector 51 and the second reflector 52 that reflect at least a portion of the light emitted by the light emitter 3 (more specifically, the light-emitting element that constitutes the light emitter 3). As illustrated in FIG. 3 and FIG. 4, according to the present embodiment, the light-guiding member 5 is arranged with the first reflector 51 and the second reflector 52 facing each other in a plan view of the light-guiding member 5 and therefore the gas sensor 1. More specifically, the light-guiding member 5 is arranged with the first reflector 51 and the second reflector 52 spaced apart in the y-axis direction (front-back direction) and facing each other in the y-axis direction (front-back direction).

As illustrated in FIG. 4, the first reflector 51 includes a first mirror 511, a third mirror 512, and a fifth mirror 513 as optical members. The first reflector 51 includes a mirror that first reflects light emitted from the light emitter 3 and a mirror that finally reflects light received by the light receiver 4. The second reflector 52 includes a second mirror 521 and a fourth mirror 522 as optical members. The light-guiding member 5 reflects light emitted by the light emitter 3 in this order from the first mirror 511, the second mirror 521, the third mirror 512, the fourth mirror 522, and the fifth mirror 513, and guides light to the light receiver 4. The optical path is configured to traverse through the cell 54 between the light-guiding member 5 and the substrate 2, where a gas body is introduced. As another example, the number of mirrors in the light-guiding member 5 is not limited to the total of five mirrors illustrated in FIG. 4, and may be two or more in total. Further, the light-guiding member 5 may be configured to include a lens or a diffractive optical element in a portion of the optical path. In this way, when light is reflected multiple times within the light-guiding member 5, the optical path length per unit volume of the light-guiding member 5 becomes longer, and therefore a small, high precision gas sensor can be realized.

In the light-guiding member 5, a position of the first reflector 51 relative to the second reflector 52 is fixed. According to the present embodiment, the light-guiding member 5 is integrally formed including the first reflector 51 and the second reflector 52. Here, integrally formed may refer to forming a plurality of members simultaneously and integrally without using secondary adhesion or mechanical joining. More specifically, according to the present embodiment, the light-guiding member 5 is formed by integrally injection molding all portions except for the mirrors using resin, and then each mirror is formed by sputtering, vapor deposition, coating or plating a metal such as aluminum. Further, each mirror may have a protective layer to protect the reflecting surface. However, as long as the light-guiding member 5 is integrally formed including the first reflector 51 and the second reflector 52, there is no limitation to this example. For example, the light-guiding member 5 may be formed by machining metal and resin or by metal press working. Further, when the light-guiding member 5 is integrally formed including a plurality of reflectors and a reflective layer is formed by coating, the coating may be applied to portions other than the reflectors in order to simplify the production process. Further, in such a case, by increasing surface roughness of a non-reflective portion other than the reflectors, reflectance of the non-reflective portion is reduced, and degradation of gas detection performance due to stray light can be suppressed.

The first mirror 511 is a light-focusing mirror that reflects light emitted from the light emitter 3 (more specifically, the light-emitting element that constitutes the light emitter 3) at a focal point. The first mirror 511 is, for example, a concave mirror. The first mirror 511 may have an ellipsoidal shape. According to the present embodiment, the first mirror 511 reflects light emitted in the z-axis direction from the light emitter 3 at the focal point in the xy plane direction. Here, the xy plane direction is a direction having a component in at least one of the x-axis direction and the y-axis direction. However, the xy plane direction may further include a z-axis direction component.

The second mirror 521, the third mirror 512, and the fourth mirror 522 reflect incident light. At least one of the second mirror 521, the third mirror 512, and the fourth mirror 522 may be a focusing mirror having a light-focusing function. At least one of the second mirror 521, the third mirror 512, and the fourth mirror 522 may be, for example, a concave mirror. As illustrated in FIG. 4, the second mirror 521 reflects light incident from the first mirror 511 to the third mirror 512. The third mirror 512 reflects light incident from the second mirror 521 to the fourth mirror 522. The fourth mirror 522 reflects light incident from the third mirror 512 to the fifth mirror 513.

The fifth mirror 513 is a focusing mirror that focuses incident light onto the light receiver 4 (more specifically, the light-receiving element that constitutes the light receiver 4). The fifth mirror 513 is, for example, a concave mirror. The fifth mirror 513 may have an ellipsoidal shape. According to the present embodiment, the fifth mirror 513 reflects incident light in the xy plane direction from the fourth mirror 522 so that the light has a component in the z-axis direction. Specifically, the fifth mirror 513 reflects incident light so that the light is focused at the light receiver 4 (more specifically, the light-receiving element that constitutes the light receiver 4) at the focal point.

Material constituting the first mirror 511, the second mirror 521, the third mirror 512, the fourth mirror 522, and the fifth mirror 513 may be, for example, metal, glass, ceramics, stainless steel, or the like, but are not limited to these examples. From the viewpoint of improving detection sensitivity, the material of these mirrors is preferably a material that has a low light absorption coefficient and high reflectance. Specifically, the light-guiding member 5 is preferably a resin housing coated with mirrors each made of an alloy containing aluminum, gold, or silver, a dielectric, or a laminate of these materials. As the resin, preferred examples include engineering plastics such as polypropylene (PP), polyamide (PA), polyphenylene ether (PPE), polycarbonate (PC), and polymethyl methacrylate (PMMA), and more preferred examples include super engineering plastics such as polyphenylene sulfide (PPS), polyether ether ketone (PEEK), and liquid crystal polymer (LCP). Further, the resin may be a mixture of the above-mentioned resins. Further, the resin may contain a filler to increase mechanical strength and reduce a coefficient of thermal expansion. Here, the filler may be, for example, glass fiber or an inorganic material. From the viewpoint of reliability and deterioration over time, the resin housing is preferably a resin housing coated with a gold or gold-containing alloy layer. Further, forming a dielectric laminate on the surface of the metal layer to increase reflectance and avoid deterioration over time is preferred. When the first mirror 511 and the fifth mirror 513 are formed by sputtering, vapor deposition, coating or plating onto a resin housing, higher productivity and lighter weight can be achieved compared to when formed from separate metal material. Further, a difference in thermal expansion coefficient from the substrate 2 is reduced, thermal deformation is suppressed, and sensitivity fluctuation is suppressed. Further, the light-guiding member 5 may be formed by machining such as cutting and milling. From the viewpoint of productivity, the light-guiding member 5 is more preferably formed by injection molding.

As illustrated in FIG. 1 and FIG. 2, according to the present embodiment, the light-guiding member 5 includes a protruding portion 53 that, when joined to the substrate 2 to form the gas sensor 1, forms a cell (a hollow portion serving as an air chamber) 54 (see FIG. 1, FIG. 4, and FIG. 5) between the light-guiding member 5 and the substrate 2 with a recess d formed therein for accommodating the light emitter 3 and the light receiver 4. The light-guiding member further includes two feet (the front foot 55 and the back foot 56) below the protruding portion 53 that are connected to the protruding portion 53. The protruding portion 53 protrudes upward from the two feet (front foot 55 and back foot 56). A lower side of the protruding portion 53 is open downward, and the recess d is formed in the protruding portion 53 to form the cell 54 when the gas sensor 1 is formed. According to the present embodiment, the external shape of the protruding portion 53 is a substantially rectangular cuboid that has a length in the front-back direction (y-axis direction) longer than a length in the left-right direction (x-axis direction), except for the open lower side. As illustrated in FIG. 1 to FIG. 3 and FIG. 5, according to the present embodiment, when the protruding portion 53 and therefore the light-guiding member 5 are joined to the substrate 2 to form the gas sensor 1, the protruding portion 53 includes a top wall 5t that faces the substrate 2 in the up-down direction (z-axis direction), a left wall 5l that is a side wall that continues to the left and below the top wall 5t, a right wall 5r that is a side wall that continues to the right and below the top wall 5t, a front wall 5f that is connected to the front side and below the top wall 5t, and a back wall 5b that is connected to the back side and below the top wall 5t. Here, the "wall" of the top wall or the like refers to a portion between an outer surface and an inner surface (including the outer surface and the inner surface), and may also be referred to as the "wall portion" hereinafter. Hereinafter, the outer surface and the inner surface of the top wall 5t, the left wall 5l, the right wall 5r, the front wall 5f and the back wall 5b may be referred to as a top outer surface 5to and a top inner surface 5ti, a left outer surface 5lo and a left inner surface 5li, a right outer surface 5ro and a right inner surface 5ri, a front outer surface 5fo and a front inner surface 5fi, and a back outer surface 5bo and a back inner surface 5bi, respectively. Further, according to the present embodiment, the light-guiding member 5 has a bottom surface 5u that can be fixed to the substrate 2 when joined to the substrate 2. The bottom surface 5u includes a left bottom surface 5lu, a right bottom surface 5ru, a front bottom surface 5fu, and a back bottom surface 5bu. According to the present embodiment, the top outer surface 5to, the left outer surface 5lo, the right outer surface 5ro, the front outer surface 5fo, the back outer surface 5bo, and the bottom surface 5u of the light-guiding member 5 are flat, but at least one of them or at least a portion of them may be curved.

As illustrated in FIG. 3, according to the present embodiment, the light-guiding member 5 includes a strain absorber 5a that can absorb strain generated in the substrate 2 and/or the light-guiding member 5. In FIG. 3, the strain absorber 5a is indicated by a large number of dots. Here, the gas sensor 1 requires that the substrate 2 and the light-guiding member 5 be firmly fixed for long-term stability. However, when the gas sensor 1 is in use, strain due to thermal expansion that may occur in the substrate 2 and/or the light-guiding member 5 (at least one of the substrate 2 and the light-guiding member 5) may cause deformation of optical surfaces of the mirrors and the like of the light-guiding member 5, which may degrade the optical characteristics of the gas sensor 1 and may also reduce gas detection performance. According to the present embodiment, by including the strain absorber 5a in the light-guiding member 5, it is possible to suppress degradation of the optical characteristics of the gas sensor 1, and in turn suppress degradation of the gas detection performance. Further, according to the present embodiment, unlike PTL 1, it is not necessary to suppress distortion of the optical path by joining the substrate and the light-guiding member only via a joining member that serves as the axis of rotation, and therefore the substrate 2 and the light-guiding member 5 can be configured to be joined robustly, and thus, when configured in this way, degradation of gas detection performance due to vibration is also suppressed. Further, when the light-guiding member 5 is formed of a material that has a relatively large thermal expansion coefficient, such as a resin, rather than a material that has a relatively small thermal expansion coefficient, such as a metal, the strain absorption effect of the strain absorber 5a becomes even greater.

According to the present embodiment, as illustrated in FIG. 3, the strain absorber 5a is provided between the first reflector 51 and the second reflector 52. In this case, strain generated in either the first reflector 51 or the second reflector 52 is prevented from being transmitted to the other, and the strain can be absorbed effectively. According to the present embodiment, the strain absorber 5a is provided approximately in the center in the y-axis direction (front-back direction) between the first reflector 51 and the second reflector 52, but is not particularly limited to this configuration. The strain absorber 5a only needs to be provided between the first reflector 51 and the second reflector 52. Further, the strain absorber 5a is preferably provided in a non-reflective portion within the light-guiding member 5. Here, the non-reflective portion refers to a region that is not a reflector such as the first reflector 51 and the second reflector 52, and a region that has a lower reflectivity than a reflector or a higher surface roughness than a reflector may also be considered a non-reflective portion.

As described above, according to the present embodiment, it is possible to suppress degradation of gas detection performance in the gas sensor due to strain.

As illustrated in FIG. 3, according to the present embodiment, the strain absorber 5a is configured as a thin portion that has a wall thickness thinner than the surrounding portion of the light-guiding member 5 around the strain absorber 5a. That is, according to the present embodiment, as illustrated schematically in FIG. 5, when the thickness (wall thickness) of the light-guiding member 5 at the strain absorber 5a is Tta and the thickness (wall thickness) of the light-guiding member 5 at the portion surrounding the strain absorber 5a is Tt, Tta < Tt. This configuration makes it possible to reliably absorb strain caused by thermal expansion in the substrate 2 and/or the light-guiding member 5. FIG. 5 is a schematic diagram for the purpose of explanation, and for example, the optical paths indicated by the long arrows are simplified versions of those illustrated in FIG. 4. When a difference in level between the strain absorber 5a and a portion surrounding the strain absorber 5a is a step amount S (= Tt - Tta), and S is 20 mm or more, the strain absorption effect is further improved. From the same viewpoint, it is preferable that the step amount S is, for example, 5 % or more of the thickness Tt. On the other hand, from the viewpoint of maintaining the strength of the light-guiding member 5 at the strain absorber 5a, the step amount S is preferably, for example, 100 mm or less, and more preferably 70 mm or less.

In the example illustrated schematically in FIG. 5, the thickness Tt and the thickness Tta are each uniform in the y-axis direction (and thus the step amount S is uniform in the y-axis direction), but at least one of the thickness Tt and the thickness Tta does not have to be uniform (and thus the step amount S does not have to be uniform). That is, as illustrated in FIG. 5, between the strain absorber 5a and the portion surrounding the strain absorber 5a, when the two boundary edges on the outer surface side of the light-guiding member 5 are indicated by the signs 5a1 and 5a2, and the two boundary edges on the inner surface side of the light-guiding member 5 are indicated by the signs 5a3 and 5a4, in the example of FIG. 5, in a cross-section view, a line extending between the boundary edge 5a1 and the boundary edge 5a2 (which is actually the inner surface of the strain absorber 5a facing in the negative z-axis direction) and a corresponding line on the outer surface of the light-guiding member 5 (in this example, the top outer surface 5to) (which is actually the outer surface of the strain absorber 5a facing in the positive z-axis direction) are straight lines extending in the y-axis direction and are parallel to a virtual straight line (indicated by a dotted line in FIG. 5) extending in the y-axis direction and connecting the boundary edge 5a3 and the boundary edge 5a4. That is, the step amount S is constant in the y-axis direction. However, in the cross-section view of FIG. 5, the line extending between the boundary edge 5a1 and the boundary edge 5a2 and/or the corresponding line on the outer surface of light-guiding member 5 may be a line other than a straight line, for example a curve, a wavy line, a jagged line, or the like, and the virtual line connecting the boundary edge 5a3 and the boundary edge 5a4 may extend diagonally with respect to the y-axis direction when the boundary edge 5a3 and the boundary edge 5a4 are positioned differently in the z-axis direction. Further, the line extending between the boundary edge 5a1 and the boundary edge 5a2 and the imaginary straight line connecting the boundary edge 5a3 and the boundary edge 5a4 do not have to be parallel to each other. That is, the step amount S does not have to be constant in the y-axis direction. In such a case, the step amount S is calculated as an average value of the distance in the z-axis direction between the line extending between the boundary edge 5a1 and the boundary edge 5a2 and the imaginary straight line connecting the boundary edge 5a3 and the boundary edge 5a4 in the cross-section view of FIG. 5. Further, at least one of the boundary edges 5a1 to 5a4 may be slightly rounded in the cross-section view of FIG. 5 due to production processing of the light-guiding member 5 and the gas sensor 1. Further, in the cross-section view of FIG. 5, the line connecting the boundary edge 5a1 and the boundary edge 5a3 does not have to be perpendicular to the line connecting the boundary edge 5a3 and the boundary edge 5a4. Similarly, the line connecting the boundary edges 5a2 and 5a4 does not have to be perpendicular to the line connecting the boundary edges 5a3 and 5a4. In order to enhance the strain absorption effect, the straight line connecting the boundary edge 5a1 and the boundary edge 5a3 is preferably nearly perpendicular to the straight line connecting the boundary edge 5a3 and the boundary edge 5a4. Similarly, the line connecting the boundary edges 5a2 and 5a4 is preferably nearly perpendicular to the line connecting the boundary edges 5a3 and 5a4. Here, "nearly perpendicular" may mean that the angle is 70° or more and less than 110°. Further, the straight line connecting the boundary edge 5a1 and the boundary edge 5a3 is preferably nearly perpendicular to the top inner surface 5ti, which is the inner surface of the top wall 5t. Similarly, the straight line connecting the boundary edges 5a2 and 5a4 is preferably nearly perpendicular to the top inner surface 5ti. Here, "nearly perpendicular" may mean that the angle is 70° or more and less than 110°.

According to the present embodiment, the strain absorber 5a is configured as a thin portion, but this is not a limitation.

As illustrated in FIG. 3 and FIG. 5, according to the present embodiment, the strain absorber 5a is provided to the top wall 5t of the light-guiding member 5. The top wall 5t extends between the first reflector 51 and the second reflector 52. Therefore, in this case, strain can be effectively absorbed. According to the present embodiment, more specifically, the thin portion serving as the strain absorber 5a is formed by recessing the top inner surface 5ti, which is the inner surface of the top wall 5t, toward the top outer surface 5to, which is the outer surface.

As illustrated in FIG. 3, according to the present embodiment, the first reflector 51 and the second reflector 52 are configured to be separated from each other by the strain absorber 5a. In other words, in the light-guiding member 5, the strain absorber 5a is interposed in all wall portions between the first reflector 51 and the second reflector 52, and as a result, the first reflector 51 and the second reflector 52 are completely separated in the y-axis direction (front-back direction) by the strain absorber 5a. More specifically, according to the present embodiment, as illustrated in FIG. 3, the strain absorber 5a is formed continuously over the entirety of the light-guiding member 5 from the top wall 5t to both side walls (left wall 5l and right wall 5r). This configuration further enhances the strain absorption effect of the strain absorber 5a.

However, as long as the strain absorber 5a is formed between the first reflector 51 and the second reflector 52, the first reflector 51 and the second reflector 52 do not have to be configured to be separated from each other by the strain absorber 5a. For example, the strain absorber 5a may be formed in a partial region of at least one of the top wall 5t, the left wall 5l, and the right wall 5r between the first reflector 51 and the second reflector 52.

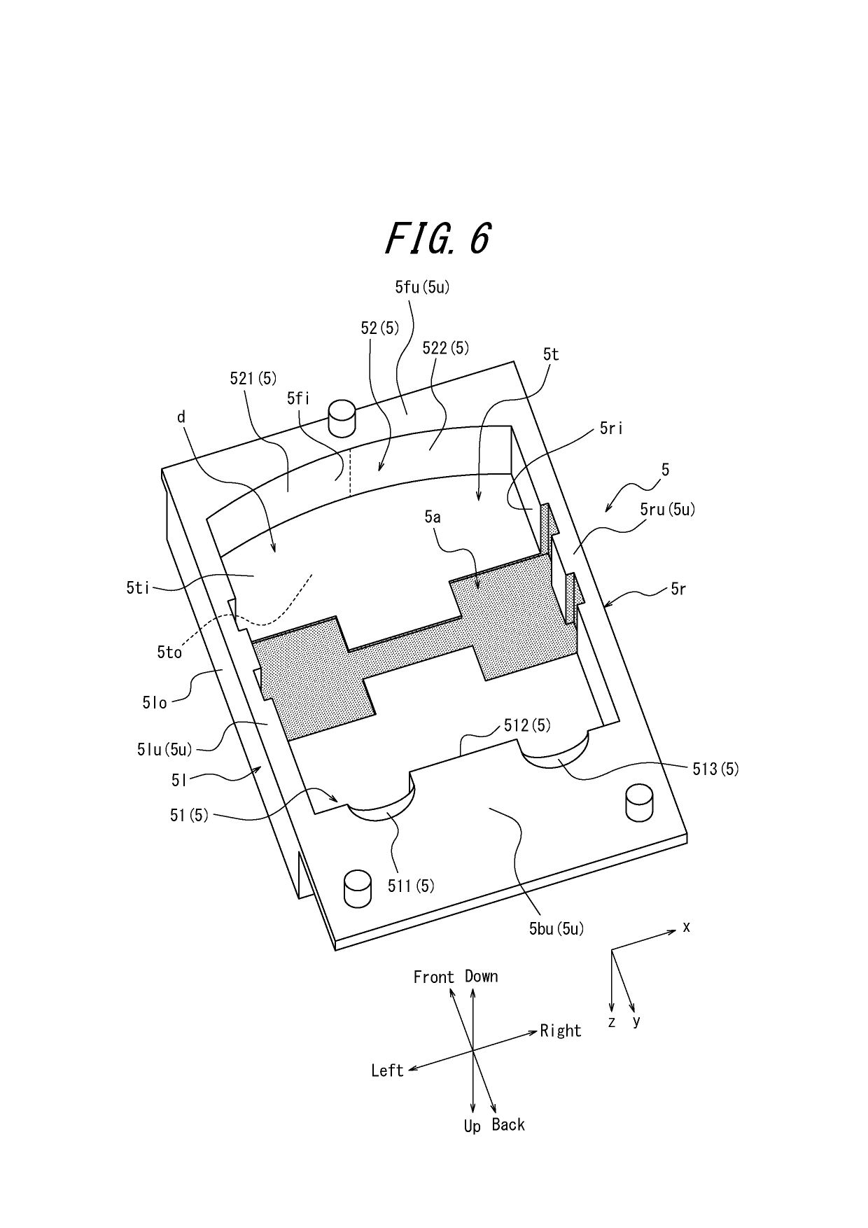

As illustrated in FIG. 3, according to the present embodiment, through holes 7a are provided in the strain absorber 5a. In this case, the through holes 7a further suppress the propagation of strain, and the strain absorption effect of the strain absorbers 5a is further enhanced. In the example of FIG. 3, two of the through holes 7a are provided in the strain absorber 5a, but the number of the through holes 7a in the strain absorber 5a may be one or three or more. The shape of each of the through holes 7a is not particularly limited. However, as in a second embodiment described later with reference to FIG. 6, the through-holes 7a do not have to be provided in the strain absorber 5a. Further, according to the present embodiment, the shape of the strain absorber 5a is a rectangle that is long in the left-right direction in plan view of the top wall 5t, excluding the portion of the through holes 7a, and each side wall (left wall 5l and right wall 5r) is a rectangle that continues the rectangular shape, but the shape of the strain absorber 5a is not particularly limited. The shape of the strain absorber 5a in plan view and/or side view may be, for example, as in the second embodiment described later.

The through holes 7a can also function as ventilation openings for introducing gas to be measured into the cell 54 (see FIG. 1, FIG. 4, and FIG. 5) formed between the light-guiding member 5 and the substrate 2, and for discharging the gas to be measured from the cell 54. When the four of the through holes 7a illustrated in FIG. 3 are viewed collectively as one ventilation opening 7, in this example, the ventilation opening 7, which is rectangular in plan view and long in the left-right direction (x-axis direction), has two beams 8: a first beam 81 extending in the short direction of the ventilation opening 7 (in this example, the y-axis direction) and a second beam 82 extending in the longitudinal direction of the ventilation opening 7 (in this example, the x-axis direction). As illustrated in FIG. 2, the first beam 81 extends toward a center O of the outer top surface 5 to of the light-guiding member 5. The beams 8 contribute to suppressing a decrease in strength and deformation of the light-guiding member 5 due to the provision of the ventilation opening 7 (and thus the through holes 7a), and thus to suppressing degradation of the optical characteristics of the light-guiding member 5. A particle filter (dust filter) may be provided on the top outer surface 5to so as to cover the entire ventilation opening 7 (and thus the through holes 7a).

Preferred configurations and variations of the gas sensor 1 of the present embodiment are further described below.

Referring to FIG. 3, according to the present embodiment, the bottom surface 5u (left bottom surface 5lu, right bottom surface 5ru, front bottom surface 5fu, and back bottom surface 5bu) of the light-guiding member 5 formed around the recess d in the protruding portion 53 of the light-guiding member 5 can serve as fixing portions between the light-guiding member 5 and the substrate 2 when the light-guiding member 5 is joined to the substrate 2. In this case, the bottom surface 5u of the light-guiding member 5 and the corresponding peripheral portion of the main surface 20 of the substrate 2 serve as the fixing portion between the light-guiding member 5 and the substrate 2.

Here, according to the present embodiment, the light-guiding member 5 and the substrate 2 are preferably fixed to each other by a plurality of fixing portions. In other words, in the example of FIG. 3, the light-guiding member 5 is preferably fixed to the substrate 2 at two or more positions on the bottom surface 5u. For example, the light-guiding member 5 is preferably fixed to the substrate 2 at one or more positions of at least two or more of the left bottom surface 5lu, the right bottom surface 5ru, the front bottom surface 5fu, and the back bottom surface 5bu. According to the above configuration, the substrate 2 and the light-guiding member 5 can be firmly joined (fixed) together while the strain absorber 5a absorbs strain, and therefore in the gas sensor 1, degradation of the gas detection performance due to strain as well as degradation of the gas detection performance due to vibration can be suppressed. In the gas sensor 1 according to the present embodiment, the light-guiding member 5 and the substrate 2 are fixed at four fixing portions, including all four positions on the bottom surface 5u of the light-guiding member 5, namely, the left bottom surface 5lu, the right bottom surface 5ru, the front bottom surface 5fu, and the back bottom surface 5bu. However, the light-guiding member 5 and the substrate 2 may be fixed to each other by a number of fixing portions other than four. According to the present embodiment, the light-guiding member 5 and the substrate 2 are fixed to each other with an adhesive at the fixing portions described above, but they may be fixed to each other by other means. The other means may be, for example, caulking, screw engagement, welding, fitting, or the like.

Further, according to the present embodiment, preferably at least two of the plurality of fixing portions are disposed so as to sandwich the strain absorber 5a when the gas sensor 1 is viewed from above. That is, in the example of FIG. 3, the light-guiding member 5 and the substrate 2 are preferably fixed together using, for example, the left bottom surface 5lu and the right bottom surface 5ru, or the front bottom surface 5fu and the back bottom surface 5bu, or all of these, which sandwich the strain absorber 5a in the light-guiding member 5, as fixing portions to the substrate 2. According to this configuration, the substrate 2 and the light-guiding member 5 can be joined (fixed) more firmly, so that degradation of gas detection performance can be more effectively suppressed.

Further, according to the present embodiment, the light-guiding member 5 is configured as follows, and can be an imaging optical system in which at least a portion of light emitted by the light emitter 3 on the substrate 2 is imaged at the light receiver 4. Similarly, in the imaging optical system of the light-guiding member 5, the size of the image formed on the light receiver 4 can be made smaller than the area of the light-receiving surface of the light receiver 4. Here, the size of the image may be the size of an area having an illuminance of 10 % or more of a peak illuminance of the image. The light-receiving surface of the light receiver 4 may be a surface of the light receiver 4 that has light-receiving sensitivity. For example, when the light receiver 4 is a photodiode chip sealed with resin, the surface of the photodiode chip onto which light is incident may be used as the light-receiving surface. Such configurations are typical when using a surface-mounted mid-infrared LED or the like as the light emitter 3 instead of a lamp, where mid-infrared LEDs have been developed and mass-produced in recent years as light sources that are significantly smaller in size than lamps that have until recently been mainly used as the light sources for gas sensors. However, when the light-guiding member 5 satisfies the above conditions, then even when there is only a slight misalignment of optical members such as mirrors or lenses or a slight deformation of the optical system, the image formed by the light receiver 4 will extend beyond the light-receiving surface of the light receiver 4, and the amount of light received by the light receiver 4 will change even though the gas concentration does not change, which will have a significant adverse effect on the optical characteristics of the gas sensor 1 and, ultimately, on the gas detection performance. Therefore, in the case described above, the effects of the various components such as the strain absorber 5a according to the present embodiment become more effective.

Further, the gas sensor 1 of the present embodiment may be of a surface mount type. That is, the gas sensor 1 may be used by being surface-mounted on a motherboard separate from the gas sensor 1, for example. In other words, the gas sensor 1 may be surface-mounted on the motherboard and used as a part of a gas sensor system including the motherboard and the gas sensor 1 surface-mounted on the motherboard. In other words, the gas sensor system may include a motherboard and the gas sensor 1 surface-mounted on the motherboard. When the gas sensor 1 is a surface-mount type, the gas sensor 1 may be heated to a maximum of approximately 260 °C in a reflow furnace during mounting. This causes significant deformation of the light-guiding member 5 due to thermal expansion, which has a significant adverse effect on the optical characteristics of the gas sensor 1 and, ultimately, on gas detection performance. Therefore, in the case described above, the effects of the various components such as the strain absorber 5a according to the present embodiment become more effective.

Next, another embodiment of the gas sensor according to the present disclosure is described with reference to the drawings.

FIG. 6 is a perspective view diagram of the light-guiding member 5 included in the gas sensor 1 according to the second embodiment of the present disclosure, viewed as in FIG. 3. The light-guiding member 5 according to the second embodiment differs from the light-guiding member 5 according to the first embodiment only in that the configuration (shape and the like) of the strain absorber 5a is different from that of the first embodiment and that the through holes 7a are not present. Other points are the same as those in the first embodiment, and therefore detailed explanations are omitted.

As illustrated in FIG. 6, the strain absorber 5a according to the second embodiment has a planar shape of the top wall 5t that is a series of, in the left-right direction, an approximate square, a rectangle whose short sides are shorter than the lengths of the sides of the approximate square, and an approximate square similar to the one on the left side. Further, in each side wall (left wall 5l and right wall 5r), the strain absorber 5a is connected to the top wall 5t and is divided into two portions in the front-back direction, each extending over the entire up-down direction of the side wall. Even with the configuration of the light-guiding member 5 and the strain absorber 5a as in the second embodiment, the strain absorbing effect of the strain absorber 5a can be obtained, and therefore degradation of the gas detection function of the gas sensor 1 can be suppressed.

Next, a simulation for confirming the effect of the gas sensor 1 according to an embodiment of the present disclosure and the results thereof are described.

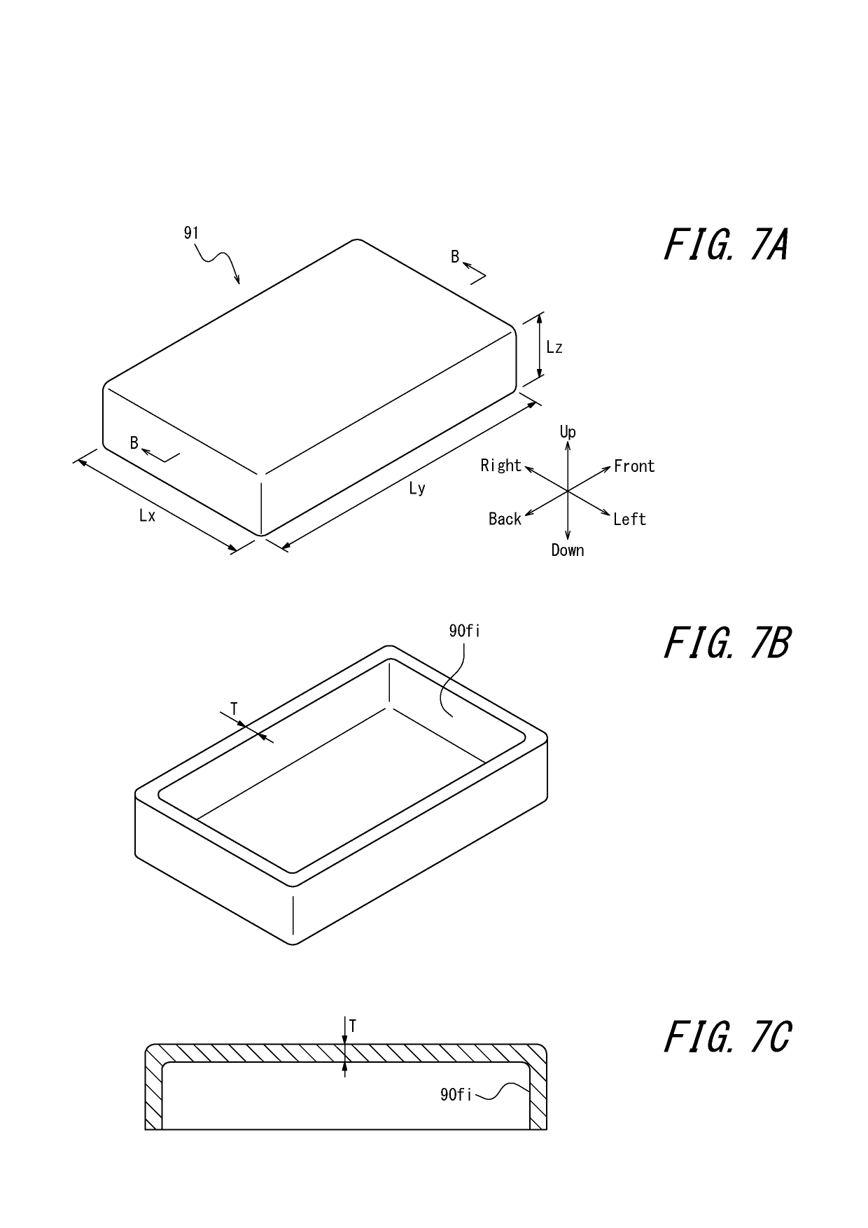

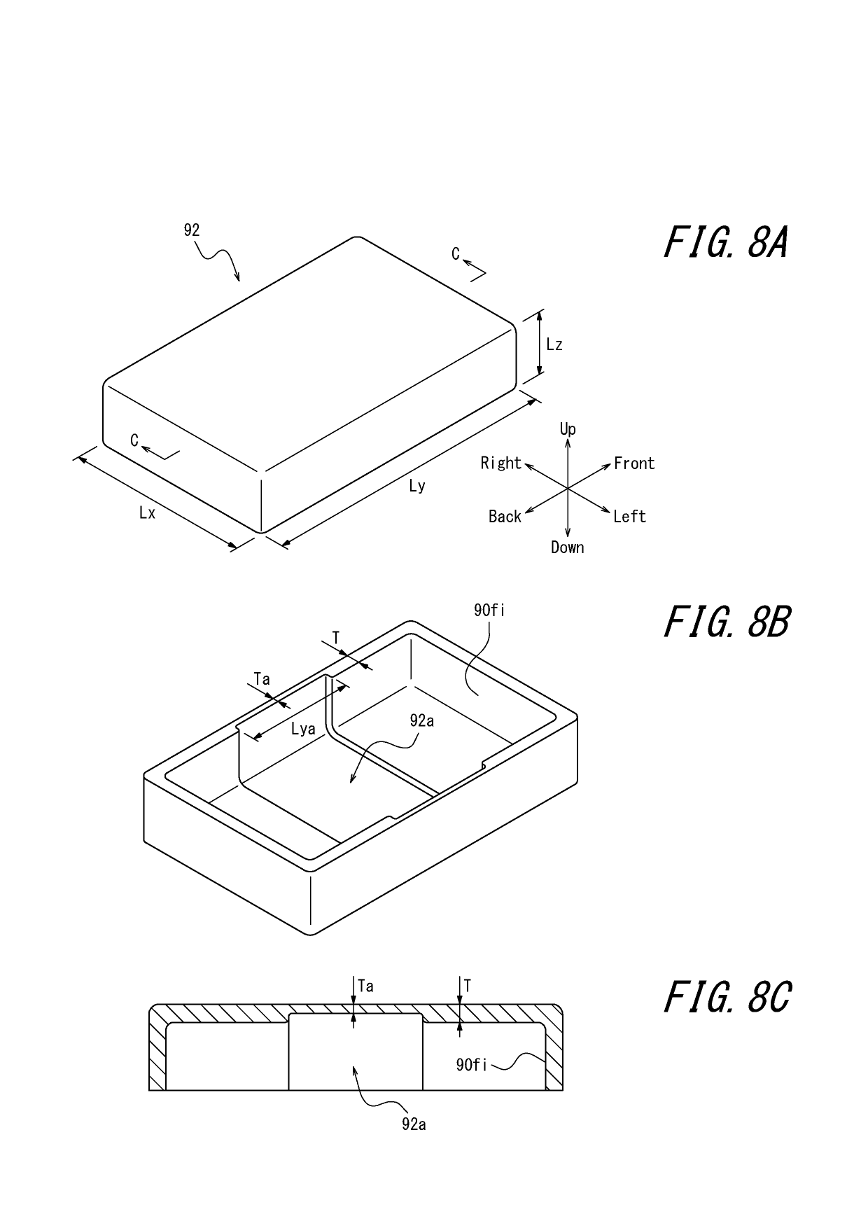

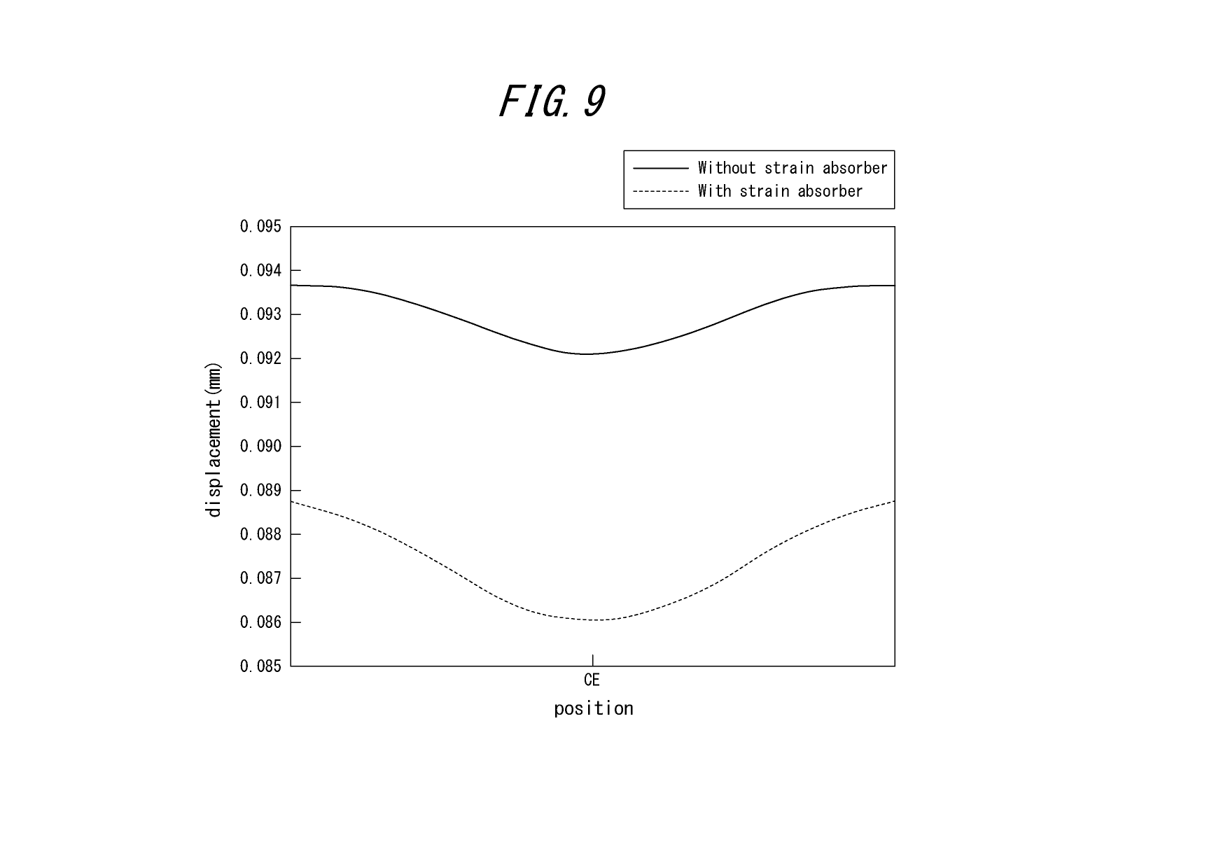

The simulation was carried out on a test article having a substantially rectangular cuboid shape having an open bottom surface, which imitated the protruding portion 53 of the light-guiding member 5 illustrated in FIG. 2 and FIG. 3. External dimensions of the test article were: front-back length Ly = 48 mm, left-right length Lx = 30 mm, and up-down length (height) Lz = 10 mm; thickness (wall thickness) T of each wall (wall portion) was constant at 2.00 mm except for a strain absorber 92a of the test article 92 of the example; and the material was assumed to be polyphenylene sulfide (PPS). However, the test article did not have a through hole corresponding to the through holes 7a in the light-guiding member 5 illustrated in FIG. 2 and FIG. 3. As illustrated in FIG. 8A to 8C, the test article 92 of the example includes the strain absorber 92a located approximately centrally in the longitudinal direction (front-back direction), extending from the top wall to both left and right walls, with a thickness (wall thickness) Ta of the top wall and both left and right walls being 1.00 mm thinner than the surrounding portions (that is, step amount T - Ta = 1.00 mm). The length Lya of the strain absorber 92a in the front-back direction was 16.00 mm, and was constant in the left-right direction. On the other hand, as illustrated in FIG. 7A to FIG. 7C, a test article 91 as a comparative example was the same as the test article 92 example except for not including a strain absorber. Then, for these test articles, the entire opening surface (bottom surface) was restrained, a temperature was increased from room temperature to 200 ° C, and a front-rear direction displacement of the front inner surface (front inner surface) 90fi of the test article, which was assumed to correspond to the second reflector 52 illustrated in FIG. 3 to FIG. 5, was determined by simulation. The results are illustrated in FIG. 9. In the graph of FIG. 9, the horizontal axis indicates the position in the left-right direction of the front inner surface 60fi of the test article, and CE indicates the center position in the left-right direction. Further, the vertical axis indicates the amount of displacement of the front inner surface 90fi of the test article in the front-to-back direction. In the graph of FIG. 9, the solid line indicates the results of the test article 91 comparative example without a strain absorber, and the dashed line indicates the results of the test article 92 example including the strain absorber 92a.

As is clear from FIG. 9, the test article 92 example including the strain absorber had a smaller overall displacement, confirming the effect of the present disclosure.

The above describes exemplary embodiments, and various modifications can be made without departing from the scope of the claims.

1. A gas sensor comprising:
a substrate comprising, on a main surface thereof, a light emitter configured to emit light and a light receiver configured to receive at least a portion of the light emitted by the light emitter; and
a light-guiding member joined to the substrate on the side of the main surface of the substrate and configured to guide at least a portion of the light emitted by the light emitter to the light receiver, wherein
the light-guiding member comprises a first reflector and a second reflector that each reflect at least a portion of the light emitted by the light emitter, and is integrally formed including the first reflector and the second reflector,
the first reflector and the second reflector are disposed to face each other in a plan view of the gas sensor, and
the light-guiding member comprises a strain absorber between the first reflector and the second reflector in the plan view, the strain absorber being capable of absorbing strain generated in the substrate and/or the light-guiding member.

2. The gas sensor according to claim 1, wherein the first reflector and the second reflector are separated from each other by the strain absorber.

3. The gas sensor according to claim 1, wherein the strain absorber is configured as a thin portion that has a wall thickness thinner than a portion of the light-guiding member surrounding the strain absorber.

4. The gas sensor according to claim 1, wherein a through hole is provided in the strain absorber.

5. The gas sensor according to claim 1, wherein a light-emitting element constituting the light emitter is an LED.

6. The gas sensor according to claim 1, wherein the light-guiding member and the substrate are fixed together by a plurality of fixing portions.

7. The gas sensor according to claim 6, wherein at least two of the plurality of fixing portions are arranged to sandwich the strain absorber in a plan view of the gas sensor.

8. The gas sensor according to claim 1, wherein the light-guiding member is an imaging optical system configured to form an image at the light receiver with at least a portion of the light emitted from the light emitter.

9. The gas sensor according to claim 8, wherein a size of the image formed by the imaging optical system of the light-guiding member is smaller than an area of a light-receiving surface of the light receiver.

10. The gas sensor according to claim 1, wherein the gas sensor is a surface mounted type.
